(19) United States
(12) Patent Application Publication
(10) Pub. No.: US 2025/0049750 A1
(43) Pub. Date: Feb. 13, 2025

(54) MALTODEXTRIN FORMULATIONS

(71) Applicant: Amazentis SA, Lausanne (CH)

(72) Inventors: Christopher L. Rinsch, Lausanne (CH); Anurag Singh, Lausanne (CH)

(21) Appl. No.: 18/799,545

(22) Filed: Aug. 9, 2024

(30) Foreign Application Priority Data
Aug. 9, 2023 (GB) 2312220.3

(51) Int. Cl. A61K 31/352; A61K 9/00; A61K 9/16; A61K 47/36

(52) U.S. Cl. CPC A61K 31/352; A61K 9/0053; A61K 9/1652; A61K 47/36

(57) ABSTRACT

Disclosed are aqueous formulations comprising urolithins particularly water-based formulations comprising urolithins and maltodextrin. Also disclosed are compositions for admixture with water, methods for the preparation of such aqueous formulations, and to the uses of such formulations for both the treatment of diseases, disorders and conditions, for example, diseases disorders or conditions characterized by inadequate mitochondrial function and for administration to generally healthy individuals for example, for improving health and improving muscle function and performance.

### RELATED APPLICATIONS

This application claims the benefit of priority to GB Patent Application No. 2312220.3, filed Aug. 9, 2023.

### BACKGROUND

Urolithins have been proposed as treatments for a variety of conditions related to inadequate mitochondrial activity, including obesity, reduced metabolic rate, metabolic syndrome, diabetes mellitus, cardiovascular disease, hyperlipidaemia, neurodegenerative diseases, cognitive disorders, mood disorders, stress, and anxiety disorders; for weight management, or to improve muscle function or mental performance (see WO2012/088519, Amazentis SA). Urolithins have also been described for the treatment of various neoplastic diseases (see WO 2007/127263, The Regents of the University of California).

Urolithins have also been disclosed for increasing autophagy, including specifically mitophagy, in a cell, comprising contacting a cell with an effective amount of a urolithin or a pharmaceutically acceptable salt thereof, thereby increasing autophagy, including specifically mitophagy, in the cell (see WO2014/004902, Amazentis SA).

As disclosed above, urolithins have been disclosed for improving muscle function. Good muscle performance is important for effective living at all stages of life in healthy individuals as well as in those individuals suffering from a disease, especially the elderly. Improved muscle performance is also of particular interest to athletes. For example, an increase in muscular contraction strength, increase in amplitude of muscle contraction, or shortening of muscle reaction time between stimulation and contraction are all of benefit to individuals, especially athletes.

Urolithins are only sparingly soluble in aqueous solutions which presents a problem for the preparation of formulations for the delivery of urolithins to subjects. However, such formulations are required to be able to administer urolithins to individuals, such as patients or healthy individuals, such as athletes.

### SUMMARY

The invention relates to aqueous formulations comprising urolithins, particularly water-based formulations comprising urolithins and maltodextrin.

The invention also relates to compositions for admixture with water, methods for the preparation of such aqueous formulations, and to the uses of such formulations, for example, for use for improving muscle function.

Surprisingly, it was found that it is possible to develop aqueous formulations of urolithins, including water dispersible compositions comprising a urolithin and maltodextrin, for example, agglomerated maltodextrin, for example for administration as beverages, for example, sports drinks.

One aspect of the invention provides a composition, comprising:

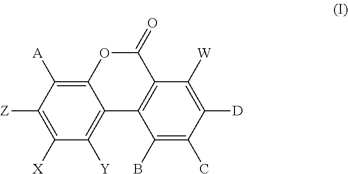

(a) a compound of formula (I), or a salt thereof; wherein A, B, C, D, W, X, Y and Z are each independently selected from H and OH; and
(b) maltodextrin, for example, agglomerated maltodextrin.

### BRIEF DESCRIPTION OF THE FIGURE

FIG. 1 shows the plasma pharmacokinetic parameters of tested maltodextrin formulation following single dose oral administration and in particular the plasma concentration of urolithin A at pre-dose and at different time points after dosing.

### DETAILED DESCRIPTION

According to a first embodiment of the invention, there is provided a composition, comprising:
(a) a compound of formula (I), or a salt thereof;

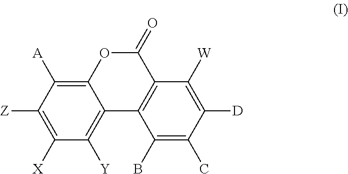

wherein:
A, B, C, D, W, X, Y and Z are each independently selected from H and OH; and
(b) maltodextrin, for example, agglomerated maltodextrin.

According to a further embodiment of the invention, there is provided a composition for admixture with water, comprising:
(a) a compound of formula (I), or a salt thereof;

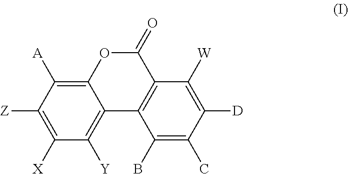

wherein:
A, B, C, D, W, X, Y and Z are each independently selected from H and OH; and
(b) maltodextrin, for example, agglomerated maltodextrin.

According to a further aspect of the invention there is provided a composition for admixture with water, comprising:
(a) a compound of formula (I), wherein the compound of formula (I) is a urolithin, for example, urolithin A, urolithin B, urolithin C or urolithin D, such as urolithin A; and
(b) maltodextrin, for example, agglomerated maltodextrin.

In a further embodiment of the invention, there is provided a composition for admixture with water, comprising:
(a) about 100 mg to about 2000 mg compound of formula (I), for example, urolithin A; and
(b) about 100 mg to about 6000 mg maltodextrin, for example, agglomerated maltodextrin.

In a further embodiment of the invention, there is provided a composition for admixture with water, comprising:
(a) about 0.4 millimoles to about 8.8 millimoles compound of formula (I), for example, urolithin A; and
(b) about 0.04 millimoles to about 7.24 millimoles maltodextrin, for example, agglomerated maltodextrin.

In a further embodiment of the invention, there is provided a composition for admixture with water, comprising:
(a) about 100 mg to about 2000 mg compound of formula (I), for example, urolithin A; and
(b) about 100 mg to about 6000 mg maltodextrin, for example, agglomerated maltodextrin.
wherein the weight ratio of a compound of Formula (I) to maltodextrin by weight is either:
(i) about 1 to 1 to about 1 to 3;
(ii) about 1 to 1.5 to about 1 to 2.5; or
(iii) about 1 to 2.5 to about 1 to 3.5.

In a further embodiment of the invention, there is provided a composition for admixture with water, comprising:
(a) about 0.4 millimoles to about 8.8 millimoles compound of formula (I), for example, urolithin A; and
(b) about 0.04 millimoles to about 7.24 millimoles maltodextrin, for example, agglomerated maltodextrin.
wherein the weight ratio of a compound of Formula (I) to maltodextrin by weight is either:
(i) about 1 to 1 to about 1 to 3;
(ii) about 1 to 1.5 to about 1 to 2.5; or
(iii) about 1 to 2.5 to about 1 to 3.5.

In a further embodiment of the invention, there is provided a composition for admixture with water, comprising:
(a) about 100 mg to about 2000 mg compound of formula (I), for example, urolithin A; and
(b) about 200 mg to about 4000 mg maltodextrin, for example, agglomerated maltodextrin.

In a further embodiment of the invention, there is provided a composition for admixture with water, comprising:
(a) about 0.4 millimoles to about 8.8 millimoles compound of formula (I), for example, urolithin A; and
(b) about 0.08 millimoles to about 4.83 millimoles maltodextrin, for example, agglomerated maltodextrin.

In a further embodiment of the invention, there is provided a composition for admixture with water, comprising:
(a) about 100 mg to about 2000 mg compound of formula (I), for example, urolithin A; and
(b) about 200 mg to about 4000 mg maltodextrin, for example, agglomerated maltodextrin;
wherein the ratio of a compound of Formula (I) to maltodextrin is about 1 to 1.5 to about 1 to 2.5 by weight.

In a further embodiment of the invention, there is provided a composition for admixture with water, comprising:
(a) about 0.4 millimoles to about 8.8 millimoles compound of formula (I), for example, urolithin A; and
(b) about 0.08 millimoles to about 4.82 millimoles maltodextrin, for example, agglomerated maltodextrin.
wherein the ratio of a compound of Formula (I) to maltodextrin is about 1 to 1.5 to about 1 to 2.5 by weight.

In a further embodiment of the invention, there is provided a composition for admixture with water, comprising:
(a) about 100 mg to about 2000 mg compound of formula (I), for example, urolithin A; and
(b) about 100 mg to about 2000 mg maltodextrin, for example, agglomerated maltodextrin.

In a further embodiment of the invention, there is provided a composition for admixture with water, comprising:
(a) about 0.4 millimoles to about 8.8 millimoles compound of formula (I), for example, urolithin A; and
(b) about 0.04 millimoles to about 2.41 millimoles maltodextrin, for example, agglomerated maltodextrin.

In a further embodiment of the invention, there is provided a composition for admixture with water, comprising:
(a) about 100 mg to about 2000 mg compound of formula (I), for example, urolithin A; and
(b) about 100 mg to about 2000 mg maltodextrin, for example, agglomerated maltodextrin.
wherein the ratio of a compound of Formula (I) to maltodextrin is about 1 to 1 to about 1 to 1.5 by weight.

In a further embodiment of the invention, there is provided a composition for admixture with water, comprising:
(a) about 0.4 millimoles to about 8.8 millimoles compound of formula (I), for example, urolithin A; and
(b) about 0.04 millimoles to about 2.41 millimoles maltodextrin, for example, agglomerated maltodextrin.
wherein the ratio of a compound of Formula (I) to maltodextrin is about 1 to 1 to about 1 to 1.5 by weight.

It would be understood by the skilled person that compositions for admixture with water, can be prepared in bulk. Therefore, compositions for admixture with water, as defined above also includes compositions comprising a multiple of the above ranges for example, composition for admixture with water, comprising:
(a) about 100 mg to about 2000 mg compound of formula (I), for example, urolithin A; and
(b) about 100 mg to about 6000 mg maltodextrin, for example, agglomerated maltodextrin, also includes compositions for admixture with water, comprising:
(a) about 1 g to about 20 g compound of formula (I), for example, urolithin A; and
(b) about 1 g to 60 g maltodextrin, for example, agglomerated maltodextrin.
and compositions for admixture with water, comprising:
(a) about 10 g to about 200 g compound of formula (I), for example, urolithin A; and
(b) about 10 g to about 600 g maltodextrin, for example, agglomerated maltodextrin.

In a further embodiment of the invention, there is provide a composition for admixture with water or an aqueous formulation comprising:
(a) about 1 part to about 20 parts by weight of a compound of formula (I), for example, urolithin A; and
(b) about 1 part to about 60 parts by weight of maltodextrin, for example, agglomerated maltodextrin.

According to a further aspect of the invention there is provided a composition for admixture with water, comprising:
(a) 1 part by weight of a compound of formula (I), or a salt thereof, for example, urolithin A; and
(b) about 1 to about 3 parts by weight maltodextrin.

According to a further aspect of the invention there is provided a composition for admixture with water, comprising:
(a) 1 part by weight of a compound of formula (I), or a salt thereof, for example, urolithin A; and
(b) about 2 to about 3 parts by weight maltodextrin.

According to a further aspect of the invention there is provided a composition for admixture with water, comprising:
(a) 1 part of a compound of formula (I), or a salt thereof, for example, urolithin A; and
(b) about 1.5 to about 2.5 parts maltodextrin.

According to a further aspect of the invention there is provided a composition for admixture with water, comprising:
(a) 1 part by weight of a compound of formula (I), or a salt thereof, for example, urolithin A; and
(b) about 1 to about 1.5 parts maltodextrin.

According to a further aspect of the invention there is provided a composition for admixture with water, comprising:
(a) 1 part by weight of a compound of formula (I), or a salt thereof, for example, urolithin A; and
(b) about 1 part by weight maltodextrin.

According to a further aspect of the invention there is provided a composition for admixture with water, comprising:
(a) 1 part by weight of a compound of formula (I), or a salt thereof, for example, urolithin A; and
(b) about 2 parts by weight maltodextrin.

According to a further aspect of the invention there is provided a composition for admixture with water, comprising:
(a) 1 part by weight of a compound of formula (I), or a salt thereof, for example, urolithin A; and
(b) about 3 parts by weight maltodextrin.

According to a further aspect of the invention, there is provided a sachet comprising a composition of the invention.

Compositions of the invention are administered in water, for ingestion by a subject, for example, an amateur or professional athlete, or an elderly subject. Compositions may be administered to a generally healthy subject, for examples, a generally healthy middle-aged or elderly subject, or to a subject suffering from a disease, disorder of condition, for example, a hospital patient. For example, compositions may be added to water, yogurts, smoothies or protein shakes. Therefore, according to a further aspect of the invention provided an aqueous formulation, comprising
(a) a compound of formula (I), or a salt thereof;

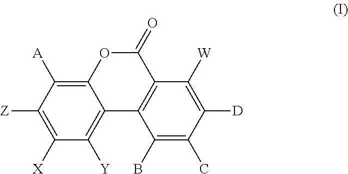

wherein:
A, B, C, D, W, X, Y and Z are each independently selected from H and OH; and
(b) maltodextrin, for example, agglomerated maltodextrin.

According to a further aspect of the invention there is provided an aqueous formulation, comprising
(a) a compound of formula (I), wherein the compound of formula (I) is a urolithin, for example, urolithin A, urolithin B, urolithin C or urolithin D, such as urolithin A; and;
(b) maltodextrin, for example, agglomerated maltodextrin.

According to a further aspect of the invention there is provided an aqueous formulation, comprising:
(a) about 100 mg to about 2000 mg of a compound of formula (I), or a salt thereof, for example, urolithin A; and
(b) about 100 mg to about 6000 mg maltodextrin.

According to a further aspect of the invention there is provided an aqueous formulation, comprising:
(a) about 100 mg to about 2000 mg of a compound of formula (I), or a salt thereof, for example, urolithin A; and
(b) about 100 mg to about 6000 mg maltodextrin,
wherein the ratio of a compound of Formula (I) to maltodextrin is about 1 to 1 to about 1 to 3.

According to a further aspect of the invention there is provided an aqueous formulation, comprising:
(a) about 0.4 millimoles to about 8.8 millimoles compound of formula (I), for example, urolithin A; and
(b) about 0.04 millimoles to about 7.24 millimoles maltodextrin, for example, agglomerated maltodextrin.

According to a further aspect of the invention there is provided an aqueous formulation, comprising:
(a) about 0.4 millimoles to about 8.8 millimoles compound of formula (I), for example, urolithin A; and
(b) about 0.04 millimoles to about 7.24 millimoles maltodextrin, for example, agglomerated maltodextrin,
wherein the ratio of a compound of Formula (I) to maltodextrin is about 1 to 1 to about 1 to 3.

According to a further aspect of the invention there is provided an aqueous formulation, comprising:
(a) about 0.4 millimoles to about 8.8 millimoles compound of formula (I); and
(b) about 0.04 millimoles to about 7.24 millimoles maltodextrin.
wherein the ratio of a compound of Formula (I) to maltodextrin is either:
(i) about 1 to 1 to about 1 to 3;
(ii) about 1 to 1.5 to about 1 to 2.5; or
(iii) about 1 to 2.5 to about 1 to 3.5.

According to a further aspect of the invention there is provided an aqueous formulation, comprising:
(a) about 100 mg to about 2000 mg of a compound of formula (I), or a salt thereof, for example, urolithin A; and
(b) about 100 mg to about 6000 mg maltodextrin.
wherein the ratio of a compound of Formula (I) to maltodextrin is about 1 to 2.5 to about 1 to 3.5.

According to a further aspect of the invention there is provided an aqueous formulation, comprising:
(a) about 100 mg to about 2000 mg of a compound of formula (I), or a salt thereof, for example, urolithin A; and
(b) about 200 mg to about 4000 mg maltodextrin.
wherein the ratio of a compound of Formula (I) to maltodextrin is about 1 to 1.5 to about 1 to 2.5.

According to a further aspect of the invention there is provided an aqueous formulation, comprising:
(a) about 100 mg to about 2000 mg of a compound of formula (I), or a salt thereof, for example, urolithin A; and
(b) about 100 mg to about 2000 mg maltodextrin.
wherein the ratio of a compound of Formula (I) to maltodextrin is about 1 to 1 to about 1 to 1.5.

According to a further aspect of the invention there is provided an aqueous formulation, comprising:
(a) 1 part by weight of a compound of formula (I), or a salt thereof, for example, urolithin A; and
(b) between about 1 to about 3 parts by weight maltodextrin.

According to a further aspect of the invention there is provided an aqueous formulation, comprising:
(a) 1 part by weight of a compound of formula (I), or a salt thereof, for example, urolithin A; and
(b) about 2 to about 3 parts by weight maltodextrin.

According to a further aspect of the invention there is provided an aqueous formulation, comprising:
(a) 1 part by weight of a compound of formula (I), or a salt thereof, for example, urolithin A; and
(b) between about 1.5 to about 2.5 part by weight maltodextrin.

According to a further aspect of the invention there is provided an aqueous formulation, comprising:
(a) 1 part by weight of a compound of formula (I), or a salt thereof, for example, urolithin A; and
(b) about 1 to about 1.5 part by weight maltodextrin.

According to a further aspect of the invention there is provided an aqueous formulation, comprising:
(a) 1 part by weight of a compound of formula (I), or a salt thereof, for example, urolithin A; and
(b) about 1 part by weight maltodextrin.

According to a further aspect of the invention there is provided an aqueous formulation, comprising:
(a) 1 part by weight of a compound of formula (I), or a salt thereof, for example, urolithin A; and
(b) about 2 parts by weight maltodextrin.

According to a further aspect of the invention there is provided an aqueous formulation, comprising:
(a) 1 part by weight of a compound of formula (I), or a salt thereof, for example, urolithin A; and
(b) about 3 parts by weight maltodextrin.

According to a further aspect of the invention, there is provided an aqueous formulation, comprising:
(a) about 25% (w/w) to about 50% (w/w) of a compound of formula (I); and
(b) about 50% (w/w) to about 75% (w/w) maltodextrin.

According to a further aspect of the invention, there is provided an aqueous formulation, comprising:
(a) about 25% (w/w) to about 50% (w/w) of a compound of formula (I); and
(b) about 50% (w/w) to about 75% (w/w) maltodextrin,
wherein the weight ratio of a compound of Formula (I) to maltodextrin is either:
(iv) about 1 to 1 to about 1 to 3;
(v) about 1 to 1.5 to about 1 to 2.5; or
(vi) about 1 to 2.5 to about 1 to 3.5.

Aqueous formulations of the invention, for ingestion by a subject, for example, an amateur or professional athlete, are for example, about 400 ml to about 1000 ml, for example, about 500 ml to about 800 ml, such as about 200 ml, about 250 ml, about 300 ml, about 400 ml, about 500 ml, about 600 ml, about 700 ml, about 800 ml, about 900 ml or about 1000 ml, such as about 400 ml to about 600 ml.

According to a further aspect of the invention there is provided an aqueous formulation, comprising:
(a) about 0.4 millimoles to about 8.8 millimoles compound of formula (I); and
(b) about 0.06 millimoles to about 3.66 millimoles maltodextrin.

According to a further aspect of the invention there is provided an aqueous formulation, comprising:
(a) about 0.4 millimolar to about 8.8 millimolar compound of formula (I); and
(b) about 0.06 millimolar to about 3.66 millimolar maltodextrin.

According to a further aspect of the invention there is provided an aqueous formulation, comprising:
(a) about 0.8 millimolar to about 17.6 millimolar compound of formula (I); and
(b) about 0.12 millimolar to about 7.32 millimolar maltodextrin.

According to a further aspect of the invention there is provided an aqueous formulation, comprising:
(a) about 1.6 millimolar to about 35.2 millimolar compound of formula (I); and
(b) about 0.24 millimolar to about 14.6 millimolar maltodextrin.

According to a further aspect of the invention there is provided an aqueous formulation, comprising:
(a) about 100 mg/litre to about 8000 mg/litre compound of formula (I); and
(b) about 100 mg/litre to about 24 g/litre maltodextrin.

According to a further aspect of the invention there is provided an aqueous formulation, comprising:
(a) about 400 mg/litre to about 8000 mg/litre compound of formula (I); and
(b) about 800 mg/litre to about 24 g/litre maltodextrin,

According to a further aspect of the invention there is provided an aqueous formulation, comprising:
(a) about 400 mg/litre to about 8000 mg/litre compound of formula (I); and
(b) about 800 mg/litre to about 24 g/litre maltodextrin.
wherein the weight ratio of a compound of Formula (I) to maltodextrin is either:
(i) about 1 to 1 to about 1 to 3 by weight;
(ii) about 1 to 1.5 to about 1 to 2.5 by weight; or
(iii) about 1 to 2.5 to about 1 to 3.5 by weight.

According to a further aspect of the invention there is provided an aqueous formulation, comprising:
(a) about 400 mg/litre to about 8000 mg/litre compound of formula (I); and
(b) about 800 mg/litre to about 16 g/litre maltodextrin,

According to a further aspect of the invention there is provided an aqueous formulation, comprising:
(a) about 400 mg/litre to about 8000 mg/litre compound of formula (I); and
(b) about 800 mg/litre to about 16 g/litre maltodextrin.
wherein the weight ratio of a compound of Formula (I) to maltodextrin is either:
(i) about 1 to 1 to about 1 to 3 by weight;
(ii) about 1 to 1.5 to about 1 to 2.5 by weight; or
(iii) about 1 to 2.5 to about 1 to 3.5 by weight.

Compositions of the invention can be added to sports drinks, for example, a bottle comprising about 500 ml to 1000 ml of a sports drink. Such sports drinks may already contain maltodextrin, for example, about 6% to about 10% (w/v) maltodextrin. Therefore, according to a further embodiment of the invention, there is provided an aqueous formulation, comprising:
(a) about 100 mg to about 2000 mg of a compound of formula (I), or a salt thereof, for example, urolithin A; and
(b) about 36.1 g to about 106 g maltodextrin.

According to a further aspect of the invention there is provided an aqueous formulation, comprising:
(a) about 0.4 millimoles to about 8.8 millimoles compound of formula (I); and
(b) about 14.7 millimoles to about 128 millimoles maltodextrin.

According to a further aspect of the invention there is provided an aqueous formulation, comprising:
(a) about 0.8 millimolar to about 17.6 millimolar compound of formula (I); and
(b) about 29.4 millimolar to about 256 millimolar maltodextrin.

According to a further aspect of the invention there is provided an aqueous formulation, comprising:
(a) about 1.6 millimolar to about 35.2 millimolar compound of formula (I); and
(b) about 58.8 millimolar to about 512 millimolar maltodextrin.

According to a further embodiment of the invention, there is provided an aqueous formulation, comprising:
(a) about 100 mg to about 2000 mg of a compound of formula (I), or a salt thereof, for example, urolithin A; and
(b) about 36.3 g to about 106 g maltodextrin.

According to a further aspect of the invention there is provided an aqueous formulation, comprising:
(a) about 0.4 millimoles to about 8.8 millimoles compound of formula (I); and
(b) about 14.8 millimoles to about 128 millimoles maltodextrin.

According to a further aspect of the invention there is provided an aqueous formulation, comprising:
(a) about 0.8 millimolar to about 17.6 millimolar compound of formula (I); and
(b) about 29.6 millimolar to about 256 millimolar maltodextrin.

According to a further aspect of the invention there is provided an aqueous formulation, comprising:
(a) about 100 mg to about 2000 mg of a compound of formula (I), or a salt thereof, for example, urolithin A; and
(b) about 30.2 g to about 104 g maltodextrin.

According to a further aspect of the invention there is provided an aqueous formulation, comprising:
(a) about 0.4 millimoles to about 8.8 millimoles compound of formula (I); and
(b) about 12.3 millimoles to about 125 millimoles maltodextrin.

According to a further aspect of the invention there is provided an aqueous formulation, comprising:
(a) about 0.8 millimolar to about 17.6 millimolar compound of formula (I); and
(b) about 24.6 millimolar to about 250 millimolar maltodextrin.

According to a further aspect of the invention there is provided an aqueous formulation, comprising:
(a) about 100 mg to about 2000 mg of a compound of formula (I), or a salt thereof, for example, urolithin A; and
(b) about 30.1 g to about 102 g maltodextrin.

According to a further aspect of the invention there is provided an aqueous formulation, comprising:
(a) about 0.4 millimoles to about 8.8 millimoles compound of formula (I); and
(a) about 12.3 millimoles to about 123 millimoles maltodextrin.

According to a further aspect of the invention there is provided an aqueous formulation, comprising:
(a) about 1.7 millimolar to about 35 millimolar compound of formula (I); and
(b) about 24.6 millimolar to about 246 millimolar maltodextrin.

According to a further aspect of the invention there is provided an aqueous formulation, comprising:
(a) about 1.7 millimolar to about 35 millimolar compound of formula (I); and
(b) about 0.244 millimolar to about 1.464 millimolar maltodextrin.

According to a further aspect of the invention there is provided an aqueous formulation, comprising:
(a) about 1.7 millimolar to about 35 millimolar compound of formula (I); and
(b) about 0.244 millimolar to about 1.464 maltodextrin.
wherein the weight ratio of a compound of Formula (I) to maltodextrin is either:
(i) about 1 to 1 to about 1 to 3;
(ii) about 1 to 1.5 to about 1 to 2.5; or
(iii) about 1 to 2.5 to about 1 to 3.5.

According to a further aspect of the invention there is provided an aqueous formulation, comprising:
(a) about 400 mg/litre to about 8000 mg/litre compound of formula (I); and
(b) about 800 mg/litre to about 24 g/litre maltodextrin,

According to a further aspect of the invention there is provided an aqueous formulation, comprising:
(a) about 400 mg/litre to about 8000 mg/litre compound of formula (I); and
(b) about 800 mg/litre to about 24 g/litre maltodextrin.
wherein the weight ratio of a compound of Formula (I) to maltodextrin is either:
(i) about 1 to 1 to about 1 to 3;
(ii) about 1 to 1.5 to about 1 to 2.5; or
(iii) about 1 to 2.5 to about 1 to 3.5.

According to a further aspect of the invention there is provided an aqueous formulation, comprising:
(a) about 400 mg/litre to about 4000 mg/litre compound of formula (I); and
(b) about 800 mg/litre to about 12 g/litre maltodextrin,

According to a further aspect of the invention there is provided an aqueous formulation, comprising:
(a) about 400 mg/litre to about 4000 mg/litre compound of formula (I); and
(b) about 800 mg/litre to about 12 g/litre maltodextrin.
wherein the weight ratio of a compound of Formula (I) to maltodextrin is either:
(i) about 1 to 1 to about 1 to 3;
(ii) about 1 to 1.5 to about 1 to 2.5; or
(iii) about 1 to 2.5 to about 1 to 3.5.

In a further embodiment, there is provided a composition for admixture with water, for example, for admixture with a sports drink comprising:
(a) about 1000 mg of a compound of formula (I), for example, urolithin A; and
(b) maltodextrin, for example, agglomerated maltodextrin, for example an agglomerated maltodextrin with a DE of about 10.

In an example embodiment, a sports drink comprises 6 litres of an aqueous solution, e.g. milk, such as oat milk, 2 kg of maltdextrin, for example, agglomerated matodextrin, and flavouring, for example, chocolate flavouring. Such a sports drink provides about 30 servings to which compositions of the invention can be added.

In another embodiment, a sports drink comprises 6 litres of an aqueous solution, e.g. 4 litres water and 2 litres of a carbonated drink, 2 kg of maltodextrin, for example, agglomerated maltodextrin, and flavouring, for example, strawberry flavouring. Such a sports drink provides about 30 servings, to which compositions of the invention can be added.

Compositions of the invention may be a powder or may be added to water, yogurts, smoothies or protein shakes. In a further embodiment of the invention, the aqueous formulation may be a shot. The term ‘shot’ refers to a concentrated beverage with a volume of less than about 100 ml, for example, with a volume in the range about 30 ml to about 90 ml.

An example, shot of the invention comprises an aqueous formulation, comprising:
(a) about 100 mg to about 1000 mg compound of Formula (I), for example, urolithin A; and
(b) about 100 mg to about 3000 mg maltodextrin in a volume of about 30 to about 90 ml, for example in a volume of about 30 ml, about 40 ml, about 50 ml, about 60 ml, about 70 ml, about 80 ml or about 90 ml.

Compositions of the invention may also be ingested in powder form.

Formulations and compositions of the invention find utility for a number of uses and in a number of indications, including improving mitochondrial function, the promotion of muscle health and performance and in the treatment of disease, disorders and conditions, particularly diseases, disorders or conditions associated with changes in muscle function.

Therefore, according to a further aspect of the invention, there is provided an aqueous formulation or composition of the invention for use in a method of increasing or maintaining mitochondrial function, for example, increasing or maintaining mitochondrial function in generally healthy subjects.

According to a further aspect of the invention, there is provided a formulation or composition of the invention for use in the treatment, prevention or management of a mitochondria-related condition associated with altered mitochondrial function or reduced mitochondrial density.

According to a further aspect of the invention, there is provided a formulation or composition of the invention for use in the treatment, prevention or management of a mitochondria-related disease.

According to a further aspect of the invention, there is provided a formulation or composition of the invention for use in increasing or maintaining mitochondrial function, for example, increasing or maintaining mitochondrial function in generally healthy subjects.

In a further embodiment of the invention there is provided a method of enhancing muscle performance comprising administering to a subject a formulation or composition of the invention. In a further embodiment, the enhanced muscle performance is selected from one or more of improved muscle function, reduced decline in muscle function, improved muscle strength, improved muscle endurance and improved muscle recovery. In a further embodiment, in the method of enhancing muscle performance, the subject suffers age-related decline in muscle function, age-related sarcopenia, age-related muscle wasting, physical fatigue, muscle fatigue, and/or is frail or pre-frail. In a further embodiment in the method of enhancing muscle performance, the subject suffers physical fatigue or muscle fatigue, and/or wherein the subject is frail or pre-frail.

In a further embodiment of the invention there is provided a method of improving, maintaining or reducing the loss of muscle function comprising administering to a subject a formulation or a composition of the invention.

In a further embodiment of the invention there is provided a method of improving endurance capacity comprising administering to a subject a formulation or composition of the invention.

In a further embodiment of the invention there is provided a method of enhancing physical performance comprising administering to a subject a formulation or a composition of the invention.

In a further embodiment of the invention there is provided a method for increasing muscle strength, increasing or maintaining muscle mass or muscle recovery comprising administering to a subject a formulation or a composition of the invention.

In a further embodiment of the invention there is provided a method of improving physical endurance comprising administering to a subject a formulation or a composition of the invention.

In a further embodiment of the invention there is provided a method of inhibiting or retarding physical fatigue, enhancing working capacity and endurance and/or reducing muscle fatigue comprising administering to a subject a formulation or a composition of the invention.

In a further embodiment of the invention there is provided a method of enhancing sports performance comprising administering to a subject a formulation or a composition of the invention.

In methods of the invention, the loss in muscle function or improvement in endurance capacity is in healthy individuals, including athletes, non-athletic individuals, sedentary individuals and the elderly.

In a further embodiment of the invention, there is provided a formulation or composition of the invention for use as a medicament.

In a further embodiment, the formulation or composition is for use as a medicament for the treatment of a muscle-related pathological condition. In a further embodiment, the muscle-related pathological condition is selected from musculoskeletal diseases or disorders; muscle wasting; myopathies; neuromuscular diseases, sarcopenia; for example, acute sarcopenia, and muscle atrophy or cachexia, or a combination of atrophy and cachexia.

In a further embodiment, the muscle atrophy or cachexia or a combination of atrophy and cachexia is selected from muscle atrophy or cachexia associated with burns, bed rest, limb immobilization, or major thoracic, abdominal, and/or orthopaedic surgery.

### Compounds of Formula (I) (Urolithins)

Compounds of formula (I) (Urolithins) are metabolites produced by the action of mammalian, including human, gut microbiota on ellagitannins and ellagic acid. Ellagitannins and ellagic acid are compounds commonly found in foods such as pomegranates, nuts and berries. Ellagitannins are minimally absorbed in the gut themselves. Urolithins are a class of compounds with the representative structure (I) shown below. The structures of some particularly common urolithins are described in Table 1 below, with reference to structure (I).

(I)

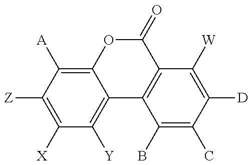

Substituent of structure (I)

| | A | B | C | D | W, X and Y | Z |
|---|---|---|---|---|---|---|
| Urolithin A | H | H | H | OH | H | OH |
| Urolithin B | H | H | H | H | H | OH |
| Urolithin C | H | H | OH | OH | H | OH |
| Urolithin D | OH | H | OH | OH | H | OH |
| Urolithin E | OH | OH | H | OH | H | OH |
| Isourolithin A | H | H | OH | H | H | OH |
| Isourolithin B | H | H | OH | H | H | H |
| Urolithin M-5 | OH | OH | OH | OH | H | OH |
| Urolithin M-6 | H | OH | OH | OH | H | OH |
| Urolithin M-7 | H | OH | H | OH | H | OH |

In practice, for commercial scale products, it is convenient to synthesise the urolithins. Routes of synthesis are described, for example, in WO 2014/004902, WO 2015/100213 and WO 2019/168972.

Urolithins of any structure according to structure (I) may be used in the formulations and compositions of the invention.

In one aspect of a formulation of composition of the invention, a suitable compound is a compound of formula (I) wherein A, C, D and Z are independently selected from H and OH and B, W, X and Y are all H, preferably at least one of A, C, D and Z is OH.

Particularly suitable compounds are the naturally-occurring urolithins. Thus, Z is preferably OH and W, X and Y are preferably all H. When W, X and Y are all H, and A, and B are both H, and C, D and Z are all OH, then the compound is Urolithin C. When W, X and Y are all H, and A, B and C are all H, and D and Z are both OH, then the compound is urolithin A. Preferably, the urolithin used in the methods of the present disclosure is urolithin A, urolithin B, urolithin C or urolithin D. Most preferably, the urolithin used is urolithin A.

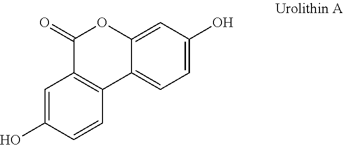 [SMILES: O=c1oc2cc(O)ccc2c2ccc(O)cc12]

According to one embodiment there is provided a formulation, composition, use or method of the invention wherein the compound of formula (I) is urolithin A.

According to one embodiment there is provided a formulation, composition, use or method of the invention wherein the compound of formula (I) is urolithin B.

According to one embodiment there is provided a formulation, composition, use or method of the invention wherein the compound of formula (I) is urolithin C.

According to one embodiment there is provided a formulation, composition, use or method of the invention wherein the compound of formula (I) is urolithin D.

In one embodiment, a compound of Formula (I) comprises acylated urolithins or optionally substituted acylated urolithins, (for example, acylated urolithin A, acylated urolithin B, acylated urolithin C, acylated urolithin D, acylated urolithin E, or acylated urolithin M5; or urolithin C having at least one hydroxyl substituted with a group containing a fatty acid). The term “acyl,” as used herein, represents a chemical substituent of formula —C(O)—R, where R is alkyl, alkenyl, aryl, arylalkyl, cycloalkyl, heterocyclyl, heterocyclyl alkyl, heteroaryl, or heteroaryl alkyl. An optionally substituted acyl is an acyl that is optionally substituted as described herein for each group R. Examples of acyl include fatty acid acyls (e.g., short chain fatty acid acyls (e.g., acetyl)) and benzoyl. In once embodiment, the acylated urolithin is a urolithin substituted by one or more C₁₋₄alkylcarbonyl groups, more example, urolithin A substituted by one or two C₁₋₄alkylcarbonyl groups (for example, formyl, acetyl or propionyl), for example, urolithin A acetate or urolithin A diacetate.

The present invention also encompasses use of suitable salts of compounds of formula (I), e.g. pharmaceutically acceptable salts. Suitable salts according to the invention include those formed with organic or inorganic bases. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine.

### Urolithin Administration/Dosage Regimes

The administration of formulations of the present invention preferably involves oral administration of a urolithin of formula (I) or salt thereof to a subject in a daily amount in the range of 1.7 to 6.0 mmol per day, for example, from about 1.7 to about 2.7 mmol per day, or from about 2.8 to about 6.0 mmol per day. As discussed below, administration is preferred in the range 250 mg to 2000 mg urolithin A (which corresponds to about 1.1 to 8.8 mmol), for example 250 mg to 1500 mg, such as 250 mg to 1000 mg, which results in a surprisingly good pharmacokinetic profile. In one embodiment the dose is 250 mg/day, in an alternative embodiment the dose is 500 mg/day and in another embodiment the dose is 1000 mg/day. In a further embodiment, the dose is 1500 mg/day. In a further embodiment, the dose is 2000 mg/day.

In a further embodiment, administration doses are selected from:
250 mg once or twice a day;
500 mg once or twice a day;
750 mg once or twice a day;
1000 mg once or twice a day;
1250 mg once or twice a day; or
1500 mg once or twice a day

The uses and methods of the present invention involve daily administration of a formulation comprising a compound of formula (I) or salt thereof. In some embodiments, the formulation is administered once per day, i.e. the formulation is to be administered at least once per 24 hour period. In other embodiments the formulation comprising the compound, is administered multiple times per day, for example twice per day, or three or four times per day. In such cases, the daily dosage is divided between those multiple doses. In one embodiment administration is once a day, in a second embodiment administration is twice a day, in a third embodiment administration is three times a day.

The methods of the present disclosure would usually require daily administration of the formulation comprising a compound of formula (I) or salt thereof, or of a formulation comprising the compound or salt, for a period over several months. In some embodiments, the methods may involve administration of the formulation comprising a compound of formula (I), or salt thereof, over for example daily for at least 2 weeks, 3 weeks, 4 weeks, 5 weeks, 6 weeks, 8 weeks, 12 weeks, 4 months, 6 months, or for at least a year. In some embodiments, the method comprises administering the formulation comprising a compound or salt thereof daily for a period of up to 3 months, up to 6 months, up to 1 year, up to 2 years or up to 5 years. In some embodiments, the method comprises administering the formulation comprising a compound or salt daily for a period in the range of from 21 days to 5 years, from 21 days to 2 years, from 21 days to 1 year, from 21 days to 6 months, from 21 days to 12 weeks, from 28 days to 5 years, from 28 days to 2 years, from 28 days to 1 year, from 28 days to 6 months, from 28 days to 4 months, from 28 days to 12 weeks, 6 weeks to 2 years, from 6 weeks to 1 year, from 8 weeks to 1 year, or from 8 weeks to 6 months.

The uses or methods of the present disclosure require daily administration of an amount of the formulation comprising a compound of formula (I) or salt thereof, of from 0.7 mmol per day up to 2.7 mmol per day thereof or from 0.7 mmol twice per day up to 2.7 mmol twice a day. In some embodiments, the amount administered is in the range of from 2.0 to 2.5 mmol. In some embodiments, the amount administered is approximately, 1.1, 1.2, 1.3, 1.4. 1.5, 1.6 1.7, 1.8, 1.9, 2.0, 2.1, 2.2, 2.3, 2.4, 2.5, 2.6, or 2.7 mmol. In some preferred embodiments the uses or method involves administration of a formulation comprising approximately 2.2 mmol per day or 2.2 mmol twice per day of the compound of formula (I) or salt thereof (e.g. of urolithin A). The exact weight of compound that is administered depends on the molecular weight of the compound that is used. For example, urolithin A has a molecular weight of 228 g/mol (such that 2.20 mmol is 501.6 mg) and urolithin B has a molecular weight of 212 g/mol (such that 2.20 mmol is 466.4 mg).

In a further embodiment, the methods of the present disclosure require daily administration of a formulation comprising an amount of compound of formula (I) or salt thereof, of from 2.8 mmol per day up to 6.0 mmol per day or twice per day thereof. In some embodiments, the amount administered is in the range of from 4.0 to 4.8 mmol. In some embodiments, the amount administered is approximately, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 mmol. In some preferred embodiments the use or method involves administration of a formulation comprising approximately 4.4 mmol per day or twice per day of the compound of formula (I) or salt thereof (e.g. of urolithin A). The exact weight of compound that is administered depends on the molecular weight of the compound that is used. For example, urolithin A has a molecular weight of 228 g/mol (such that 4.40 mmol is 1003.2 mg) and urolithin B has a molecular weight of 212 g/mol (such that 4.40 mmol is 932.8 mg).

In some embodiments the methods involve administration of a formulation comprising urolithin A in an amount in the range of from 400 to 600 mg/day or 400 to 600 mg twice per day. In a preferred embodiment the method involves administration of urolithin A in an amount in the range of from 450 to 550 mg, more preferably approximately 500 mg per day or twice per day.

In other embodiments the methods involve administration of a formulation comprising urolithin A in an amount in the range of from 700 to 1300 mg/day twice per day, or in the range of from 750 to 1250 mg, or in the range of from 800 to 1200 mg, or in the range of from 850 to 1150 mg, or in the range of from 900 to 1100 mg per day or twice per day. In a preferred embodiment the method involves administration of urolithin A in an amount in the range of from 950 to 1150 mg/day or twice per day, more preferably approximately 1000 mg/day or twice per day.

In some preferred embodiments, the uses or methods involve administering a formulation comprising urolithin A to the subject in an amount in the range of from 4.5 to 11 mg/kg/day, such as 4.5 to 8.5 mg/kg/day. In another embodiment, the uses or methods involve administering a formulation comprising urolithin A to the subject in an amount in the range of 5 to 9 mg/kg/day. In another embodiment, the uses or methods involve administering a formulation comprising urolithin A to the subject in an amount in the range of from 6.0 to 8 mg/kg/day.

In other preferred embodiments, the uses or methods involve administering a formulation comprising urolithin A to the subject in an amount in the range of from 9 to 18 mg/kg/day such as 9 to 17 mg/kg/day. In another embodiment, the uses or methods involve administering or a formulation comprising urolithin A to the subject in an amount in the range of from 10 to 17 mg/kg/day. In another embodiment, the uses or methods involve administering a formulation comprising urolithin A to the subject in an amount in the range of from 11 to 16 mg/kg/day.

Dosage regimes which combine a 500 mg dose and a 1000 mg dose may be advantageous. For example, a twice daily dosage regime which combines a first dose of 1000 mg and a second dose several hours later of 500 mg. Said 500 mg dose may be 6-18 hours after the 1000 mg dose, for example 8-12 hours after the 1000 mg dose. For example, about 12 hours after the 1000 mg dose. Thus, according to a further aspect of the invention there is provided the treatment of a disease, disorder or condition with a formulation comprising a compound of Formula (I) which comprises a twice daily dosage regime comprising a first dose of 1000 mg, followed by a second dose of 500 mg wherein the two doses are separated by 6-18 hours.

The formulation comprising a compound of formula (I) or salt thereof, may be administered at any suitable time, for example, it may be administered in the morning after sleep or in the evening. In some embodiments, it may be preferable for the method to be performed at approximately the same time(s) each day, for example within 15, 30, 60 or 120 minutes of a given time point.

Formulations comprising a compound of Formula (I) and maltodextrin can be administered before, after or during exercise. Such formulation would find utility for both amateur and professional athletes.

For the avoidance of doubt in this application, where there is reference to ‘a urolithin’, this means one or more urolithins, for example, a mixture of urolithin A and urolithin B.

### Maltodextrin

Maltodextrin is a polysaccharide consisting of a polymer of D-glucose units of variable length, typically composed of a mixture of chain varying from 3 to 20 glucose units long. Maltodextrins are classified by DE (dextrose equivalent) and have a DE between 3 and 20. The higher the DE value, the shorter the glucose chains, the higher the sweetness, the higher the solubility, and the lower the heat resistance. Maltodextrins for use in formulations and compositions of the invention typically comprise maltodextrin with a DE between about 1 and about 20, for example, about 1 to about 15, for example about 5 to about 15, for example, about 7 to about 13, for example, about 8 to about 12, for example, about 9 to about 11, for example, about 10.

In an embodiment of the invention, the maltodextrin is agglomerated maltodextrin.

Aqueous formulations of the invention comprise between 200 mg and 6000 mg maltodextrin, for example, agglomerated urolithin A.

In a further embodiment of the invention, unit doses of aqueous formulation of the invention comprise about 0.04 millimoles to about 0.724 millimoles maltodextrin.

In a further embodiment of the invention, aqueous formulations of the invention comprise maltodextrin at a concentration of about 0.163 millimolar to about 2.896 millimolar maltodextrin.

In a further embodiment of the invention, aqueous formulations comprise a urolithin and maltodextrin at a ratio of about 1:1 to about 1:3 of urolithin to maltodextrin by weight. In one embodiment, aqueous formulations comprise a comprise a urolithin and maltodextrin in a ratio of about 1 to about 1 urolithin to maltodextrin by weight. In a further embodiment, aqueous formulations comprise a comprise a urolithin and maltodextrin in a ratio of about 1 to about 2 urolithin to maltodextrin by weight. In a further embodiment, aqueous formulations comprise a comprise a urolithin and maltodextrin in a ratio of about 1.5 to about 2.5 urolithin to maltodextrin by weight. In a further embodiment, aqueous formulations comprise a comprise a urolithin and maltodextrin in a ratio of about 1 to about 3 urolithin to maltodextrin by weight. In a further embodiment, aqueous formulations comprise a comprise a urolithin and maltodextrin in a ratio of about 2.5 to about 3.5 urolithin to maltodextrin by weight.

In a further embodiment of the invention, a composition of the invention, for admixture with water comprises maltodextrin at a concentration of about 0.04 millimoles to about 0.724 millimoles maltodextrin, or multiples thereof.

### Additional/Combination Therapy

Compositions of the invention may comprise one of more further active agents, for use in the treatment or prevention of diseases, disorders or conditions or for use in the promotion of muscle health and performance in healthy individuals, or for improving mitochondrial function, or for the treatment of other conditions disclosed in the present application, for medical treatments or administration to healthy individuals.

Any active agent which is known to be useful, or which has been used or is currently being used for the treatment or prevention of diseases, disorders or conditions or for use in the promotion of muscle health and performance in healthy individuals, or for improving mitochondrial function can be used with a combination of the invention. See, e.g., Gilman et al, Goodman and Gilman's: The Pharmacological Basis of Therapeutics, 13th ed., McGraw-Hill, New York, 2017; The Merck Manual of Diagnosis and Therapy, Robert S. Porter, M. D. et al. (eds.), 20th Ed., Merck Sharp & Dohme Research Laboratories, Rahway, NJ, 2018; Cecil Textbook of Medicine, 25th Ed., Goldman and Schafer (eds.), Elsevier, 2015, and Physicians' Desk Reference (71st ed. 2016) for information regarding therapies (e.g., prophylactic or therapeutic agents) which have been or are currently being used.

Examples of further agents include, but not limited to, caffeine, omega-3 polyunsaturated fatty acids, resveratrol, spermidine, pyrroloquinoline, quinone, genistein, hydroxyltyrosol, L-creatine, L-arginine, curcumin, vitamin D, quercetin, trehalose, anti-oxidants (such as L-carnitine, coenzyme Q₁₀, MitoQ₁₀ and other mitochondria-targeted antioxidants), N-acetylcysteine (NAC), vitamin C, vitamin E, vitamin K1, one or more B vitamins (e.g thiamine, folate), B50 or B100 (B vitamin complexes), sodium pyruvate and α-lipoic acid), NAD precursors (for example, niacin, niacinamide, leucovorin and amino acids.

### Uses

Compositions and formulations of the invention can be used for both therapeutic and non-therapeutic uses, finding use in the treatment of various diseases as well as health conditions not considered to be a disease. In particular, disease conditions may be characterised by an inadequate mitochondrial activity and non-disease health improvements may be characterised by enhancements of mitochondrial function. Therefore, according to one embodiment of the invention there is provided a composition of the invention for use as a medicament for the treatment of a disease disorder or condition. According to a further embodiment of the invention there is provided a non-therapeutic method of use of a composition of the invention.

The compositions of the invention can be used as a dietary supplement, as a functional food and as a medical food. The compositions of the invention can be used for food for special medical purposes.

Compositions of the invention find utility in the treatment of disease, disorders and conditions. Therefore, according to a further aspect of the invention, there is provided a composition of the invention for use as a medicament for the treatment of a disease disorder or condition.

According to a further aspect of the invention there is provided a composition, of the invention for use in the treatment, prevention or management of a mitochondria-related condition associated with altered mitochondrial function or reduced mitochondrial density.

According to a further aspect of the invention there is provided a non-therapeutic method for preventing or managing a mitochondria-related condition associated with altered mitochondrial function (such as decreased mitochondrial function including due to the presence of fewer mitochondria and/or the presence of damaged mitochondria) or reduced mitochondrial density, comprising administration of an effective amount of a composition of the invention.

According to a further aspect of the invention there is provided a formulation or composition of the invention for use in the treatment, prevention or management of a mitochondria-related disease associated with altered mitochondrial function or reduced mitochondrial density.

According to a further aspect of the invention there is provided a composition, of the invention for use in the treatment, prevention or management of a mitochondria-related disease.

According to a further aspect of the invention there is provided a formulation or composition of the invention for use in increasing or maintaining mitochondrial function.

According to a further aspect of the invention there is provided a non-therapeutic method of increasing or maintaining mitochondrial function, comprising administration of an effective amount of a composition of the invention.

According to a further aspect of the invention there is provided a composition, of the invention for use in the treatment or prevention of inflammation.

### Therapeutic Uses

### Neurological Disorders

According to one embodiment of the invention, there is provided a composition of the invention for use in the treatment of neurological disorders, including neurodegenerative disorders. Examples of neurological disorders include: cognitive decline, memory decline, anxiety, depression, epilepsy, stroke, amyotrophic lateral sclerosis, dementia (e.g. vascular dementia and Alzheimer's disease), Parkinson's disease. and Huntington disease.

According to a further embodiment of the invention, there is provided a composition of the invention for use in the prophylaxis or treatment of a disease state initiated or characterized (i) by a decline in cognitive function; or (ii) by mood disturbances. Such disease states can include, without limitation, neurodegenerative disease, cognitive disorder, mood disorder and anxiety disorder.

An embodiment of the invention is a method of improving cognitive function. The method includes the step of administering to a subject in need thereof an effective amount of a urolithin or a precursor thereof, to improve cognitive function. In one embodiment, the cognitive function is selected from the group consisting of perception, memory, attention, speech comprehension, speech generation, reading comprehension, creation of imagery, learning, and reasoning. In one embodiment, the cognitive function is selected from the group consisting of perception, memory, attention, and reasoning. In one embodiment, the cognitive function is memory.

According to a further embodiment, there is provided a composition of the invention for use for protecting against brain oxidative stress.

According to a further embodiment, there is provided a composition of the invention for use for reducing brain inflammation.

According to a further embodiment, there is provided a composition of the invention for use for treating mood disorders, stress and/or anxiety disorders.

### Immune Function

In a further embodiment of the invention there is provided the use of a composition of the invention for supporting and/or enhancing immune function during or after cancer treatment.

In a further embodiment of the invention there is provided the use of a composition of the invention for supporting and/or enhancing immune function remission after cancer treatment.

In a further embodiment of the invention there is provided the use of a composition of the invention for supporting and/or enhancing immune function during and after hospital admissions.

### Aging/Healthspan

According to a further embodiment of the invention, there is provided a composition of the invention for use in increasing healthspan.

In a further embodiment of the invention, there is provided a composition of the invention, for slowing aging.

According to a further embodiment of the invention there is provided a composition of the invention for increasing longevity.

In one embodiment, healthspan is said to be increased when it is at least 5 percent longer than placebo control or an untreated group. In various specific embodiments, healthspan is said to be increased when it is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 percent longer than placebo control or untreated group. In certain embodiments, healthspan is increased by at least 10 percent compared to placebo control or an untreated group. In certain embodiments, healthspan is increased by at least 20 percent compared to placebo control or an untreated group. In certain embodiments, healthspan is increased by at least 30 percent compared to placebo control or an untreated group. In certain embodiments, healthspan is increased by at least 40 percent compared to placebo control or an untreated group. In certain embodiments, healthspan is increased by at least 50 percent compared to placebo control or an untreated group.

### Skin

In a further embodiment of the invention, there is provided a composition of the invention for use in a method of prevention, management and/or treatment of a skin condition, disease or disorder.

In a further embodiment of the invention, there is provided a composition of the invention for use in the treatment of a skin condition, disease or disorder is a skin condition, disease or disorder associated with inadequate mitochondrial activity.

In some embodiments, the skin disease, disorder or condition is selected from the group consisting of melasma, chloasma, hyperpigmentation, skin-aging, liver spots, lentigo, inflammation of the skin, skin irritation, skin infection, warts, psoriasis, and protection of skin from damage caused by the environment and/or therapy. The skin disease, disorder or condition is also selected from melanosis, dermatitis, linea nigra and endocrine diseases such as Addison's and Cushing's syndrome.

As mentioned above, the composition of the invention may be for use in a method of prevention of a skin condition, disease or disorder. That is to say that it is suitable for use by subjects with skin prone to the ailments mentioned herein, for example subjects with intermittent skin issues, for example, skin prone to sunburn, prone to acne and prone to inflammation. The compositions finds use in the management and prevention of the conditions.

In a further embodiment of the invention, there is provided a composition of the invention for the treatment of a condition selected from acne, eczema, psoriasis and rosacea.

In a further embodiment of the invention, there is provided a composition of the invention for the prevention and treatment of skin prone to condition selected from acne, eczema, psoriasis and rosacea.

### Treatments:

The composition of the invention can be taken as a single treatment or, more commonly, as a series of treatments. In one example, a subject takes a dose before or after exercise. For a subject who is not able to exercise, a dose of the composition may, for example, be taken once, twice or three times per day, or one, two, three, four, five or six times per week. In another example, the intervention may be taken by a subject independent of the subject's ability or need to exercise. It will also be appreciated that the effective dosage of the compound may increase or decrease over the course of a particular treatment.

Recovery after Treatment

Composition of the invention may also be used in patients recovering from a disease, disorder or condition, whether at home or in hospital. Therefore, according to a further embodiment of the invention there is provided a composition of the invention for use in aiding recovery of a patient from a disease, disorder or condition, for example, a disease selected from cancer, bacterial or viral infection or other serious illness, or a physical trauma, such as a car accident.

### Non-Therapeutic Uses

Compositions of the invention find use as nutritional supplements, functional foods, medical nutrition and equivalents. These include treatments of non-therapeutic conditions, i.e. treatment of conditions in generally healthy subjects.

### Muscle Function, Performance, Endurance

The composition finds use in the management of normal physiological function in healthy individuals of conditions characterised by poor physical performance, impaired endurance capacity, and impaired muscle function. Compositions of the invention may improve physical performance in individuals with a disease, including young and elderly individuals. Compositions of the invention may improve physical performance, for example, short-term performance or long-term performance in healthy individuals, including athletes, non-athletic individuals, sedentary individuals and the elderly. This improvement of performance may be measured by the time spent to walk or run a certain distance (for example, an improved performance during the 6 minute walk test (MWT)), an improved time to run a certain distance, an improved IPAQ score on the international physical activity questionnaire, an increased number of chair-stands in a certain time, or another test designed to measure physical performance.

Compositions of the invention further provide for the improvement of endurance capacity. The endurance capacity refers to the time to fatigue when exercising at a constant workload, generally at an intensity <80% VO₂ₘₐₓ. Compositions of the invention may improve endurance capacity in individuals with a disease, including young and elderly individuals. Compositions of the invention may improve endurance capacity in healthy individuals, including athletes, non-athletic individuals, sedentary individuals and the elderly. The invention provides for a method of increasing the time to fatigue while performing a specific activity, for example, fitness training, walking, running, swimming, or cycling. This improvement of endurance capacity may be assessed with objective measurements (for example, speed, oxygen consumption or heart rate) or it can be self-reported measurements (for example, using a validated questionnaire).

The invention further provides a composition to improve, maintain or reduce the loss of muscle function. Compositions of the invention may improve, maintain or reduce the loss of muscle function in individuals with a disease, including young and elderly individuals. Compositions of the invention may improve, maintain or reduce the loss of muscle function in healthy individuals, including athletes, non-athletic individuals, sedentary individuals and the elderly. For example, compositions of the invention may increase muscle strength as evidenced by the improvement of performing a physical activity, such as an exercise, for example, increased ability to lift weights or increased hand grip strength. Also, compositions of the invention may improve muscle structure, for example by increasing or maintaining muscle mass in conditions of normal muscle function, declining muscle function or impaired muscle function.

This invention further provides a composition to improve the physical performance or endurance capacity as perceived by the individual. For example, by the reduction of in perceived exertion or effort during exercise or an activity as determined using a self-reported questionnaire.

### Immune Health

In a further embodiment of the invention there is provided the use of a composition of the invention for maintaining or enhancing immune health.

In a further embodiment of the invention there is provided the use of a composition of the invention for reducing or slowing inflammaging.

In a further embodiment of the invention there is provided the use of a composition of the invention for slowing immune aging.

In a further embodiment of the invention there is provided the use of a composition of the invention for reducing immune cell aging.

In a further embodiment of the invention there is provided the use of a composition of the invention for boosting immune function.

In a further embodiment of the invention there is provided the use of a composition of the invention for supporting a healthy immune system.

In a further embodiment of the invention there is provided the use of a composition of the invention for prevention or management of colds and/or flu.

In a further embodiment of the invention there is provided the use of a composition of the invention for the treatment of infection.

In a further embodiment of the invention there is provided the use of a composition of the invention for improving vaccination response.

In a further embodiment of the invention there is provided the use of a composition of the invention for enhancing an immune response against infection.

In a further embodiment of the invention there is provided the use of a composition of the invention for protecting against infection, for example, protection against colds and flu.

In a further embodiment of the invention there is provided the use of a composition of the invention for immune support.

In a further embodiment of the invention there is provided the use of a composition of the invention for reducing the risk of respiratory tract infections (for example, colds, flu or pneumonia).

In a further embodiment of the invention there is provided the use of a composition of the invention for increasing the rate of recovery from respiratory tract infections (for example, colds, flu or pneumonia).

In a further embodiment of the invention there is provided the use of a composition of the invention for increasing CD8 positive T-cells, for example, naïve CD8-positive T-cells.

### Brain Health

According to a further embodiment of the invention there is provided the use of a composition of the invention for maintaining or enhancing brain health.

According to a further embodiment of the invention there is provided the use of a composition of the invention for promoting healthy brain function.

According to a further embodiment, there is provided a composition of the invention for use for optimising brain health.

According to a further embodiment, there is provided a composition of the invention for use to improve, protect, and maintain brain function and cognition.

According to a further embodiment, there is provided a composition of the invention for use for enhancing mental alertness, for example, mental accuracy and clearer/sharper thinking.

According to a further embodiment, there is provided a composition of the invention for use for increasing psychomotor speed and/or reaction time.

According to a further embodiment, there is provided a composition of the invention for use for enhancing/improving memory.

According to a further embodiment, there is provided a composition of the invention for use for enhancing/improving learning.

According to a further embodiment, there is provided a composition of the invention for use for enhancing/improving reasoning, for example, promoting mental processing.

According to a further embodiment, there is provided a composition of the invention for use for enhancing/improving focus, for example, concentration, complex attention and/or sustained attention.

According to a further embodiment, there is provided the use of a composition of the invention for stress relief and/or anxiety relief.

According to a further embodiment, there is provided the use of a composition of the invention for treating stress and/or anxiety.

According to a further embodiment, there is provided the use of a composition of the invention for treating depression.

According to a further embodiment, there is provided the use of a composition of the invention for improving mood.

According to a further embodiment, there is provided the use of a composition of the invention for enhancing sleep and/or relaxation.

### Skin

In a further embodiment of the invention, there is provided a composition of the invention for use to manage or maintain skin health and/or reduce the impact of chronological aging on biological aging. In a further there is provided use of a composition of the invention for prevention or management of aging skin.

In a further embodiment of the invention, there is provided the use of composition of the invention for the maintaining or enhancing skin health in a generally healthy subject.

According to a further embodiment of the invention, there is provided use of a composition of the invention for one or more of the following:
a) improving skin health;
b) for maintaining or enhancing skin energy;
c) for maintaining or enhancing skin collagen;
d) reducing skin biological aging;
e) supporting healthy skin aging;
f) reducing skin wrinkles, and/or fine lines;
g) improving skin mitochondrial function;
h) improving skin hydration;
i) promoting replenishment of skin ceramide levels;
j) improving skin elasticity and barrier function;
k) protecting against free radical damage; and/or
I) improving skin dryness;
m) improving photodamaged skin;
n) Improving skin health in skin prone to redness, inflammation, acne or dryness.

According to a further embodiment of the invention, there is provided use of a composition of the invention for one or more of the following:
a). maintaining or enhancing skin health and appearance;
b). maintaining or improving skin hydration;
c). enhancing skin elasticity;
d). reducing wrinkles and fine lines;
e). supporting healthy skin aging;
f). helping replenish ceramide stores;
g). helping protect against free radical damage;
h). support for sun-damage
i). use as an antioxidant;
j). maintaining or promoting healthy skin, hair and nails; and/or
k). skin barrier support.

### Hair

In a further embodiment of the invention, there is provided a composition of the invention for use to manage or maintain hair health. In a further embodiment of the invention, there is provided a composition of the invention for example, a composition comprising urolithin A, for one or more of the following uses:
(a) maintaining or enhancing hair thickness;
(b) hair follicle cell regeneration;
(c) hair follicle cell survival;
(d) hair stem cell growth and/or regeneration;
(e) hair cell survival;
(f) hair loss prevention,
(g) hair maintenance;
(h) scalp health improvement,
(i) improving or maintaining hair strength, and/or
(j) hair growth restoration promotion.

In a further embodiment of the invention, there is provided a composition of the invention for example, a composition comprising urolithin A, for one or more of the following uses:
a) maintaining or enhancing hair thickness;
b) hair follicle cell regeneration;
c) hair follicle cell survival;
d) hair stem cell growth and/or regeneration;
e) hair cell survival;
f) hair loss prevention,
g) scalp health improvement, and/or
h) hair growth restoration promotion

In a further embodiment there is provided a method for:
a) maintaining or enhancing hair thickness;
b) hair follicle cell regeneration;
c) hair follicle cell survival;
d) hair stem cell growth and/or regeneration;
e) hair cell survival;
f) hair loss prevention,
g) promoting new hair growth;
h) scalp health improvement,
i) for improving survival of hair transplants; and/or
j) hair growth restoration promotion.

In a further embodiment of the invention, there is provided a composition of the invention for example, a composition comprising urolithin A, for one or more of the following uses:
a) enhancing hair stem cell function;
b) enhancing hair follicle elongation,
c) enhancing hair matrix proliferation, for example, as measured by Ki67 levels;
d) enhancing hair growing phase, (enhancing the duration of the anaphase);
e) hair matrix proliferation,
f) inhibition of hair matrix apoptosis; and
g) reducing melanin clumping, for example, in pigmentary units, for example, in hair follicle pigmentary units.

In a further embodiment of the invention, there is provided a composition of the invention, for example, a composition comprising urolithin A, for one or more of the following uses:
a) improving hair healthspan; and/or
b) improving hair longevity;

### Muscle Performance

The composition of the invention is useful in enhancing muscle performance. The invention thus provides a composition of the invention for use in enhancing muscle performance. The invention also provides a method of enhancing muscle performance by administering to a subject an effective amount of a composition of the invention. Administration can be self-administration. The enhanced muscle performance may be one or more improved muscle function, improved muscle strength, improved muscle endurance and improved muscle recovery.

The composition of the invention can thus be used in a method of improving physical endurance (e.g., ability to perform a physical task such as exercise, physical labor, sports activities), inhibiting or retarding physical fatigue, enhancing working capacity and endurance, reducing muscle fatigue, enhancing cardiac and cardiovascular function.

Improved muscle function can be particularly beneficial in elderly subjects with reduced muscle function as a result of an age-related condition. For example, a subject who may benefit from improved muscle function may experience a decline in muscle function which then leads to pre-frailty and frailty. Such subjects may not necessarily experience muscle wastage in addition to their decline in muscle function. Some subjects do experience both muscle wasting and a decline in muscle function, for example subjects with sarcopenia. The composition of the invention may be used in enhancing muscle performance by administering a composition of the invention to a subject who is frail or pre-frail.

Muscle performance may be sports performance, which is to say the ability of an athlete's muscles to perform when participating in sports activities. Enhanced sports performance, strength, speed, and endurance are measured by an increase in muscular contraction strength, increase in amplitude of muscle contraction, or shortening of muscle reaction time between stimulation and contraction. Athlete refers to an individual who participates in sports at any level and who seeks to achieve an improved level of strength, speed, or endurance in their performance, such as, for example, body builders, bicyclists, long distance runners, and short distance runners. Enhanced sports performance is manifested by the ability to overcome muscle fatigue, ability to maintain activity for longer periods of time, and have a more effective workout.

### Muscle Uses of Formulations and Compositions of the Invention

The formulations and compositions of the invention find use in improving muscle performance, improving muscle function, preventing a decline in muscle function, increasing muscle mass and/or reducing muscle wasting. The improvement in muscle performance, improving or maintaining muscle function, the increase in muscle mass and/or reduction in muscle wasting may be as part of a medical treatment, or it may be for personal preference (“lifestyle”) or cosmetic reasons. The formulations and compositions of the invention can be for use as a medicament. The formulations and compositions can be used as a dietary supplement, as a functional food, functional beverage, or as a medical food. The enhanced muscle performance may be one or more improved muscle function, improved muscle strength, improved muscle endurance and improved muscle recovery.

The formulations and compositions find use in the treatment of both diseases and disease states. The formulations and compositions find use in the management normal physiological function in healthy individuals in conditions characterised by poor physical performance, impaired endurance capacity, and impaired muscle function. Formulations and compositions of the invention may improve physical performance in individuals with a disease or generally healthy individuals, including young and elderly individuals. Formulations and compositions of the invention may improve physical performance, for example, short-term performance or long-term performance in healthy individuals, including athletes, non-athletic individuals, sedentary individuals and the elderly. This improvement of performance may be measured by the time spent to walk or run a certain distance (for example, an improved performance during the 6 minute walk test (MWT)), an improved time to run a certain distance, an improved IPAQ score on the international physical activity questionnaire, an increased number of chair-stands in a certain time, or another test designed to measure physical performance.

Formulations and compositions of the invention further provide for the improvement of endurance capacity. The endurance capacity refers to the time to fatigue when exercising at a constant workload, generally at an intensity <80% VO₂ₘₐₓ. Formulations and compositions of the invention may improve endurance capacity in individuals with a disease, including young and elderly individuals. Formulations and compositions of the invention may improve endurance capacity in healthy individuals, including athletes, non-athletic individuals, sedentary individuals and the elderly. The invention provides for a method of increasing the time to fatigue while performing a specific activity, for example, fitness training, walking, running, swimming, or cycling. This improvement of endurance capacity may be assessed with objective measurements (for example, speed, oxygen consumption or heart rate) or it can be self-reported measurements (for example, using a validated questionnaire).

The invention further provides a formulation or composition to improve muscle function, maintain muscle function, reduce the loss of muscle function or reduce muscle inflammation post exercise, or enhance muscle recovery post exercise. Formulations and compositions of the invention may improve, maintain or reduce the loss of muscle function in individuals with a disease, including young and elderly individuals. Formulations and compositions of the invention may improve, maintain or reduce the loss of muscle function in healthy individuals, including athletes, non-athletic individuals, sedentary individuals and the elderly. For example, formulations or compositions of the invention may improve, maintain or reduce the loss of muscle function in frail or pre-frail individuals. For example, formulations or compositions of the invention may increase muscle strength as evidenced by the improvement of performing a physical activity, such as an exercise, for example, increased ability to lift weights or increased hand grip strength. Also, formulations or compositions of the invention may improve muscle structure, for example by increasing or maintaining muscle mass in conditions of normal muscle function, declining muscle function or impaired muscle function.

This invention further provides a formulation or composition to improve the physical performance or endurance capacity as perceived by the individual. For example, by the reduction of in perceived exertion or effort during exercise or an activity as determined using a self-reported questionnaire.

### Methods of Enhancing Physical Performance

According to a further embodiment of the invention, there is provided a composition or formulation of the invention for use in a method of enhancing physical performance in a subject, for example, in an elite athlete or sub-elite athlete.

In certain embodiments, enhancing physical performance comprises at least one effect selected from the group consisting of enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage. In further embodiments, enhancing physical performance comprises enhancing muscle performance during a high-intensity aerobic activity. In yet further embodiments, enhancing physical performance comprises increasing aerobic endurance during a high-intensity aerobic activity. In still further embodiments, enhancing physical performance comprises increasing resting metabolic rate (RMR). In certain embodiments, enhancing physical performance results in an improvement in athletic performance. In further embodiments, enhancing physical performance results in an improvement in footrace completion times. In yet further embodiments, enhancing physical performance results in a decrease in Ratings of Perceived Exertion (RPE).

In one embodiment, the enhancing physical performance comprises enhancing muscle performance during a high-intensity aerobic activity.

In one embodiment, the enhancing physical performance comprises increasing aerobic endurance during a high-intensity aerobic activity.

In one embodiment, the enhancing physical performance comprises increasing resting metabolic rate (RMR).

In one embodiment, the enhancing physical performance results in an improvement in athletic performance.

In one embodiment, the enhancing physical performance results in an improvement in footrace completion times.

In one embodiment, the enhancing physical performance results in a decrease in Ratings of Perceived Exertion (RPE).

### Methods of Enhancing Physical Recovery

According to a further embodiment of the invention, there is provided a composition or formulation of the invention for use in a method of enhancing physical recovery in a subject, for example, in an elite athlete or sub-elite athlete.

In certain embodiments, physical recovery is enhanced after a high-intensity aerobic activity. In further embodiments, enhancing physical recovery comprises at least one effect selected from the group consisting of enhancing muscle recovery, enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage. In yet further embodiments, enhancing physical recovery comprises enhancing muscle recovery after a high-intensity aerobic activity. In still further embodiments, enhancing physical recovery comprises reducing muscle soreness after a high-intensity aerobic activity. In certain embodiments, enhancing physical recovery comprises lowering creatine kinase (CK) levels in the subject, compared to baseline, following an aerobic activity as measured by area under the plasma concentration-time curve of CK (AUC_{CK}). In further embodiments, enhancing recovery comprises lowering C-reactive protein (CRP) levels in the subject, compared to baseline, following an aerobic activity as measured by area under the plasma concentration-time curve (AUC_{CRP}).

In one embodiment, the enhancement of physical recovery is after high intensity aerobic exercise.

In one embodiment, the enhancing physical recovery comprises enhancing muscle recovery after a high-intensity aerobic activity.

In one embodiment, the enhancing physical recovery comprises reducing muscle soreness after a high-intensity aerobic activity.

In one embodiment, the enhancing physical recovery comprises lowering creatine kinase (CK) levels in the subject, compared to baseline, following an aerobic activity as measured by area under the plasma concentration-time curve of CK (AUC_{CK}).

In one embodiment, the enhancing recovery comprises lowering C-reactive protein (CRP) levels in the subject, compared to baseline, following an aerobic activity as measured by area under the plasma concentration-time curve (AUC_{CRP}).

### Methods of Enhancing Physical Endurance

According to a further embodiment of the invention, there is provided a composition or formulation of the invention for use in a method of enhancing endurance in a subject, for example, in an elite athlete or sub-elite athlete.

In certain embodiments, the method comprises enhancing physical endurance during a high-intensity aerobic activity. In further embodiments, enhancing physical endurance comprises at least one effect selected from the group consisting of enhancing muscle recovery, enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage. In yet further embodiments, enhancing physical endurance comprises enhancing muscle endurance. In still further embodiments, enhancing physical endurance comprises increasing maximal oxygen consumption (VO₂ₘₐₓ) in the subject.

In one embodiment, the enhancement of physical recovery is during high intensity aerobic exercise.

In one embodiment, the enhancing physical endurance comprises enhancing muscle endurance.

In one embodiment, the enhancing physical endurance comprises increasing maximal oxygen consumption (VO2max) in the subject.

In certain embodiments, methods of the present disclosure comprise administering to a subject in need thereof an effective amount of a formulation or composition of the invention, wherein the subject is an elite athlete or sub-elite athlete. In some such embodiments, the subject is an elite athlete. In certain embodiments, the subject has a VO₂ₘₐₓ of greater than 65 mL kg-1 min-1. In further embodiments, the subject has a 3 km running personal best time below 9 minutes. In other embodiments, the subject is a sub-elite athlete. In certain such embodiments, the subject has a VO₂ₘₐₓ of from about 60 mL kg-1 min-1 to about 65 mL kg-1 min-1. In further embodiments, the subject has a 3 km running personal best time from about 9 minutes to about 10 minutes. In certain embodiments, the age of the subject is from about 18 years to about 45 years.

### Medical Treatments:

The formulation or composition of the invention can be for use as a medicament or a medical food. The formulation or compositions of the invention find use in the treatment of muscle-related pathological conditions. Accordingly, the invention provides a formulation or composition of the invention for use in the treatment of a muscle-related pathological condition. The invention also provides a method of treating a muscle-related pathological condition in a subject comprising administering to the subject an effective amount of a formulation or composition of the invention. Muscle-related pathological conditions include both conditions impacting generally healthy individuals as well as pathological conditions. Such muscle conditions found in healthy people or people affected by a disease include musculoskeletal diseases or disorders; cachexia; muscle wasting; myopathies; age-related decline in muscle function; pre-frailty; frailty; neuromuscular diseases, such as Duchenne muscular dystrophy and other dystrophies; sarcopenia, for example, acute sarcopenia; muscle atrophy and/or cachexia, for example muscle atrophy and/or cachexia associated with burns, bed rest, limb immobilization, or major thoracic, abdominal, and/or orthopedic surgery; and muscle degenerative disease.

Examples of age-related disease conditions that may be treated with formulation or compositions of the invention include sarcopenia and muscle wasting.

### Muscle Performance:

The formulation or composition of the invention is useful in enhancing muscle performance. The invention thus provides a formulation or composition of the invention for use in enhancing muscle performance. The invention also provides a method of enhancing muscle performance by administering to a subject an effective amount of a formulation or composition of the invention. Administration can be self-administration.

The enhanced muscle performance may be one or more improved muscle function, reduced decline in muscle function, improved muscle strength, improved muscle endurance and improved muscle recovery.

The formulation or composition of the invention can thus be used in a method of improving physical endurance (e.g., ability to perform a physical task such as exercise, physical labour, sports activities), inhibiting or retarding physical fatigue, enhancing working capacity and endurance, and reducing muscle fatigue.

Improved muscle function can be particularly beneficial in elderly subjects with reduced muscle function as a result of an age-related condition. For example, a subject who may benefit from improved muscle function may experience a decline in muscle function which then leads to pre-frailty and frailty. Such subjects may not necessarily experience muscle wastage in addition to their decline in muscle function. Some subjects do experience both muscle wasting and a decline in muscle function, for example subjects with sarcopenia. The formulation or composition of the invention may be used in enhancing muscle performance by administering a composition of the invention to a subject who is frail or pre-frail.

Muscle performance may be sports performance, which is to say the ability of an athlete's muscles to perform when participating in sports activities. Enhanced sports performance, strength, speed, and endurance are measured by an increase in muscular contraction strength, increase in amplitude of muscle contraction, or shortening of muscle reaction time between stimulation and contraction. Athlete refers to an individual who participates in sports at any level and who seeks to achieve an improved level of strength, speed, or endurance in their performance, such as, for example, body builders, cyclists, long distance runners, and short distance runners. Enhanced sports performance is manifested by the ability to overcome muscle fatigue, ability to maintain activity for longer periods of time, and have a more effective workout.

### Animals

Compositions of the invention are suitable for other mammals in addition to humans, scaled appropriately to the size of the non-human mammal. For example, scaled according to the following table;

| | | To convert | To convert human dose in mg/ | |
|---|---|---|---|---|
| | Reference | dose in mg/kg | kg to AED in mg/kg, either | |
| | body | to dose | Multiply | Divide |
| | weight | in mg/m², | human | human |
| Species | (kg) | divide by km | dose by | dose by |
| Mouse | 0.02 | 3 | 12.3 | 0.081 |
| Hamster | 0.08 | 5 | 7.4 | 0.135 |
| Rat | 0.15 | 6 | 6.2 | 0.162 |
| Ferret | 0.3 | 7 | 5.3 | 0.189 |
| Guinea Pig | 0.4 | 8 | 4.6 | 0.216 |
| Rabbit | 1.8 | 12 | 3.1 | 0.324 |
| Cat | 2 | 11.7 | 3.2 | 0.316 |
| Monkey | 3 | 12 | 3.1 | 0.324 |
| Dog | 10 | 20 | 1.9 | 0.541 |
| Marmoset | 0.35 | 6 | 6.2 | 0.162 |
| Squirrel | 0.6 | 7 | 5.3 | 0.189 |
| Monkey | | | | |
| Baboon | 12 | 20 | 1.9 | 0.541 |
| Micro pig | 12 | 27 | 1.4 | 0.73 |
| Mini pig | 40 | 35 | 1.1 | 0.946 |

Therefore, according to a further aspect of the invention there is provided a composition for use in a non-human mammal wherein the dose is scaled according to the table above.

### Formulations and Compositions

The uses and methods of the present invention preferably involve oral administration of the aqueous formulation of the invention. Aqueous formulation typically include further excipients. Accordingly, the formulation or composition is administered in the form of an oral formulation and one or more excipients suitable for oral administration. Oral compositions are typically provided in powder form or as a tablet for dispersion in an aqueous medium, for example, water, milk or an energy or sports drink.

In a further embodiment of the invention, the formulation of the invention is administered by any means known to the skilled person for administration such as, intramuscular, sublingual, cutaneous, and auricular. Oral administration is preferred.

Aqueous formulations may be in the form of a medicine, a medical food, a dietary supplement, or a beverage, each for oral consumption. Aqueous formulations may be solutions, emulsions, slurries or other semi-liquids. Excipients in a liquid composition can, for example, provide a shelf-life, visual appearance, flavour and mouth-feel such that the composition has an acceptable taste, an attractive appearance and good storage stability. At certain levels of dilution, a drink may need to be shaken before the subject drinks it, so as to maintain an even suspension of the active ingredient.

In some preferred embodiments, the use or method comprises administration of a formulation comprising a compound of formula (I) or salt thereof (e.g. urolithin A), in micronized form. Micronization enables the compound of formula (I) to disperse or dissolve more rapidly. Micronisation can be achieved by methods established in the art, for example compressive force milling, hamermilling, universal or pin milling, or jet milling (for example spiral jet milling or fluidised-bed jet milling) may be used. Jet milling is especially suitable. If micronized compound is used, then preferably the compound has a D₅₀ size of under 100 μm—that is to say that 50% of the compound by mass has a particle diameter size of under 100 μm. More preferably, the compound has a D₅₀ size of under 75 μm, for example under 50 μm, for example under 25 μm, for example under 20 μm, for example under 10 μm. More preferably, the compound has a D₅₀ in the range 0.5-50 μm, for example 0.5 to 20 μm, for example 0.5 to 10 μm, for example 1.0 to 10 μm, for example 1.5 to 7.5 μm, for example 2.8 to 5.5 μm. Preferably, the compound has a D₉₀ size of under 100 μm. More preferably, the compound has a Do size of under 75 μm, for example under 50 μm, for example under 25 μm, for example under 20 μm, for example under 15 μm. The compound preferably has a D₉₀ in the range 5 to 100 μm, for example 5 to 50 μm, for example 5 to 20 μm, for example 7.5 to 15 μm, for example 8.2 to 16.0 μm. Preferably, the compound has a D₁₀ in the range 0.5-1.0 μm. Preferably, the compound of formula (I) or salt thereof (e.g. urolithin A) has a D₉₀ in the range 8.2 to 16.0 μm, a Do in the range 2.8 to 5.5 μm and a D₁₀ in the range 0.5 to 1.0 μm.

In a further embodiment, the compound of formula (I) or salt thereof has a size distribution selected from one of the following:
(i) Do size in the range 0.5 to 50 μm and a D₉₀ size in the range 5 to 100 μm,
(ii) the compound has a D₉₀ size in the range 8.2 to 16.0 μm, a D₅₀ size in the range 2.8 to 5.5 μm and a D₁₀ size in the range 0.5 to 1.0 μm;
(iii) the compound of Formula (I) has a D₅₀ size in the range 0.5 to 20 μm and a D₉₀ size in the range 5 to 50 μm;
(iv) the compound of Formula (I) has a D₅₀ size under 50 μm and a D₉₀ size under 75 μm;
(v) the compound of Formula (I) has a D₅₀ size under 25 μm and a D₉₀ size under 50 μm;
(iv) the compound of Formula (I) has a D₅₀ size under 10 μm and a D₉₀ size under 20 μm;
(v) the compound of Formula (I) has a D₅₀ size under 10 μm and a D₉₀ size under 15 μm; or
(vi) the compound of Formula (I) has a D₅₀ size of 10 μm and a D₉₀ size of 20 μm.

The term ‘about’ refers to a tolerance of ±20% of the relevant value, for example ±15% of the relevant value, such as ±10% of the relevant value or ±5% of the relevant value.

The term “elite” athlete may refer to a human subject who has a VO₂ₘₐₓ of greater than 65 mL Kg⁻¹ min⁻¹, a 3 km running personal best time below 9 minutes, or both.

The term ‘excipient’ refers to a substance formulated alongside the active ingredient, included, for example, for the purpose of long-term stabilization, bulking up solid formulations that contain potent active ingredients in small amounts (thus often referred to as “bulking agents”, “fillers”, or “diluents”), or to confer a therapeutic enhancement on the active ingredient in the final dosage form, such as facilitating absorption, reducing viscosity or enhancing solubility.

The term ‘healthspan’ refers to refers to the period of an individual's life during which they are generally healthy and free from serious or chronic illness.

The term “longevity” as used herein refers to the lifespan of an individual organism, for example, a human.

The term “pharmaceutically acceptable” means approved by a regulatory agency of the Federal or a state government or listed in the U.S. Pharmacopeia or other generally recognized pharmacopeia for use in animals, and more particularly in humans.

The term “sub-elite” athlete may refer to a human subject who has a VO₂ₘₐₓ from 60 mL Kg⁻¹ min⁻¹ to 65 mL Kg⁻¹ min⁻¹, a 3 km running personal best time from 9 to 10 minutes, or both.

The term “therapeutically effective amount” as used herein refers to the amount of a compound or compounds that, when administered, is sufficient to prevent the development of, or to relieve to some degree, one or more symptoms of the disease that it targets. The particular dose of each compound administered according to this invention will of course be determined by the particular conditions surrounding the case, including the compound administered, the route of administration, the particular condition being treated, as well as considerations such as age, weight and sex of the treated subject.

In the avoidance of doubt when the text refers to a ratio between X and Y, the ratio includes the exact value of X and Y in addition to just after X and just before Y.

For avoidance of doubt, where this description of the invention refers to uses of compositions of the invention, the description is also referring to use of formulations of the invention.

### EQUIVALENTS

The invention has been described broadly and generically herein. Those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

The invention will now be illustrated with respect to the following non-limiting examples

### Example 1: Urolithin A Solubility Tests

### Methodology

Experiments were performed to find an ingredient that can aid in the solubility of Urolithin A, testing 3 different ingredients that can be identified as candidate carriers, Urolithin A was mixed in different ratios to find the best dry mix that is soluble in water, as set out below.

The following carriers were tested:
Carrier 1: Maltodextrin DE 10 Maltrin M100
Carrier 2: Maltodextrin DE 10 Maltrin QD M500 (Agglomerated)
Carrier 3: Organic Gum Acacia—TICORGANIC Arabic Spray Dry Powder

### Using the Following Ratios:

| | Urolithin A (%) | Carrier (%) |
|---|---|---|
| Test 1 | 66.67 | 33.33 |
| Test 2 | 50 | 50 |
| Test 3 | 33.33 | 66.67 |

### Experiments Used 1 g Urolithin a Per Serving as Follows:

| | Urolithin A (g) | Carrier (g) | Total Dry Mix (g) |
|---|---|---|---|
| Test 1 | 1 | 0.5 | 1.5 |
| Test 2 | 1 | 1 | 2 |
| Test 3 | 1 | 2 | 3 |

In the experiments, the dry mix was mixed in 200 mL of room temperature water (22° C.).

### Results:

| | Result |
|---|---|
| Carrier 1 | |
| Test 1 | Hard to stir in, some lump is feasible on the |
| | surface of water |
| Test 2 | Better to stir in, some lumps is feasible |
| Test 3 | Better to stir in, least lumps from carrier 1 tests |
| Carrier 2 | |
| Test 1 | Good but still has some lumps |
| Test 2 | Good and after stirring in has no lump |
| Test 3 | Very easy to stir in and no lump of dry mix on the |
| | surface of water. Best result of carrier 2 tests. |
| Carrier 3 | |
| Test 1 | Very difficult to stir in, very lumpy and even after |
| | stirring in for a while it doesn't go in |
| Test 2 | Very difficult to stir in, many lumps on the |
| | surface of water |
| Test 3 | Many lumps on the surface of water |

### Results and Discussion

Matrin QD M500 has the best performance in solubility when mixed 2:1 ratio with Urolithin A. It is designed for dry mix blend for instant beverage and it performs very well. Maltrin M100 can be used in a dry mix but requires a lot of stirring and it will still have some lump on the surface of the water. Gum acacia is not designed to be carrier of dry mix blend and is very difficult to work with.

### Example 2: Phase II Study to Determine the Bioavailability of Urolithin a when Dosed in a Maltodextrin Formulation to Human Subjects

A phase II clinical study was carried out to determine the bioavailability of urolithin A when dosed in a formulation containing maltodextrin to human subjects.

In particular, the pharmacokinetic parameters of the compound were investigated in an open-label, parallel-arm, randomized, single-period, single-dose pharmacokinetic study. Subject enrolled where healthy volunteers, both males and females aged between 18 and 45 years. Formulation details and clinical study procedures are described in the method section below.

Subjects were administered orally as a single dose with a maltodextrin formulation (Table 2). The formulations was administered to deliver a total dose of 500 mg Urolithin A per dosing. Plasma samples were collected pre-dosing and at sequential time points after dosing, i.e. 1 hr, 4 hrs, 6 hrs, 8 hrs, 12 hrs, 24 hrs, 72 hrs. Plasma levels of parent Urolithin A were determined and expressed as pg/ml.

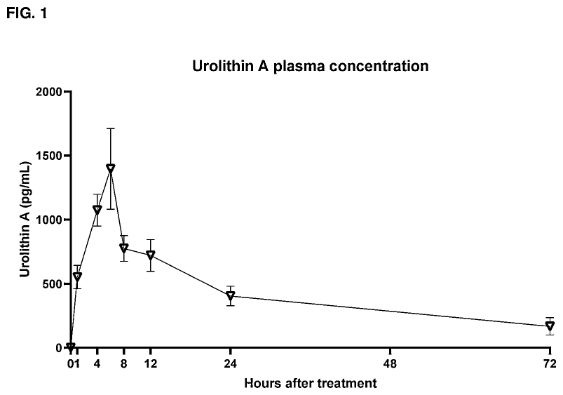

Surprisingly the maltodextrin formulation shows a surprisingly good pharmacokinetic profile. Table 1 and FIG. 1 show the pharmacokinetic results showing that the formulation containing maltodextrin is highly bioavailable. Tmax was at 6 hours, Cmax was 1397 μg/ml, and the total AUC (area under the curve) was 1283 μg/mL/hr.

TABLE 1
Urolithin concentrations in blood (mean ±
standard deviation), n = 12 subjects

| | Mean | |
|---|---|---|
| Time | (pg/mL) | SD |
| Pre | 1.00 | 3.46 |
| 1 h | 551.50 | 314.67 |
| 4 h | 1073.55 | 430.11 |
| 6 h | 1396.55 | 1091.37 |
| 8 h | 774.92 | 348.76 |
| 12 h | 720.08 | 428.70 |
| 24 h | 403.50 | 262.10 |
| 72 h | 166.99 | 235.45 |

### Methods

### Study Participants

A total of 24 participants were enrolled in the study (12 subject per group). Subjects were healthy male and female participants. Subjects were selected after a screening to meet all the following criteria aged between 18 and 45 years; with Body Mass Index (BMI) between 18.50-30.00 kg/m2;

### Test Products Information and Dosing

Maltodextrin formulation (urolithin A Powder with Maltodextrin): half a scoop (1.5 g) of powder, containing 500 mg of Urolithin A. Composition is described in Table 2.

Mode of Administration: Participants take the powder orally on an empty stomach in a sitting position, after an overnight fast. The powder was mixed with 240±2 mL of milk, shaken well, and consumed. It was ensured that all liquid was swallowed without leaving any residue behind or retaining in the mouth.

TABLE 2
Ingredients of Investigational Product- Pro Athletes
Muscle recovery powder with Maltodextrin

| | mg/serving | |
|---|---|---|
| Ingredients | (One full scoop) | % Formula |
| Urolithin A | 1000.000 | 36.90% |
| IP Maltodextrin M500 | 1870.000 | 62.10% |
| (H)Syloid Silicon Dioxide 244 | 30.000 | 1.00% |
| | | 100.00% |

### Food and Fluid Intake

Fasting for at least 12 hours before dosing until at least 04 hours post-dose. Participants were kept on standardized fluid intake during the study. The fluid intake was maintained at approximately between 3 to 3.5 liters for up to 24 hours to ensure an adequate urine flow rate from housing day till ambulatory visit day. Water was restricted from at least 1 hour before dosing until at least 1-hour post-dose (no fluid, except water given with dosing).

During the study period, participants consumed only the food provided while confined in the clinical facility (hospital). All participants were served a standard meal (Pure veg food balanced diet of protein, carbohydrates, and fat recommended by the hospital nutritionist) at least 04 hours after dosing. Further meals/snacks after 04 hours post dose were served at appropriate times.

Participants were prohibited from consuming foods rich in Urolithin A precursors from the at least seven days prior to beginning of their stay in the housing facility until the end of the ambulatory visit. These foods include fruits such as pomegranates, peaches, persimmons, and plums, as well as nuts like walnuts, hazelnuts, pistachios, acorns, chestnuts, pecans, almonds, and peanuts. Additionally, participants avoided berries such as strawberries, raspberries, blackberries, blueberries, cloudberry, and camucamu, along with muscadine grapes, oak-aged wines and spirits, and medicinal plants and tisanes (such as geranium and oak leaves), as well as their derivatives like juices, jams, and jellies.

The participants were advised to refrain from consuming dietary supplements that could potentially impact either muscle or mitochondrial function, such as resveratrol, pomegranate and ellagitannins, nicotinamide riboside, whey protein, leucine, iso-leucine, I-carnitine, creatinine, coenzyme Q10, vitamin A, niacin, folic acids, vitamin C, vitamin E and probiotic foods and supplements, during the 2 weeks before inclusion and throughout the study.

In a further embodiment, compositions and formulations of the invention further comprise electrolytes.

### Clinical Study Methodology

This study was an open-label, parallel-arm, single-period, single-dose, randomized study wherein each participant received a single dose of allocated investigational product administered orally to the trial participants in a sitting posture. The formulation was administered with about 240 mL of whole milk (3% fat content) in a sitting posture.

### Study Duration

Screening Assessments (Day −29 to −2)
Day −1: Housing
Day 01: Administration (Day of Dosing)/PK sample collection
Day 02: PK sample collection/Discharge from study site.
Day 04: PK sample collection/End of study

The trial was conducted in accordance with the Declaration of Helsinki (Ethical Principles for Medical Research Involving Human Subjects, 64th World Medical Association-General Assembly, Fortaleza, Brazil, October 2013), ICH-GCP, AYUSH Good Clinical Practice, New Drugs and Clinical Trials Rules, 14 Oct. 2022, Ministry of Health and Family Welfare, Government of India, Indian Council of Medical Research (Ethical Guidelines for Biomedical Research on Human Participants, 2017) and other applicable regulatory requirements.

### Blood Sampling for Pharmacokinetics

PK blood samples were collected at pre-dose and post-dose at 1 hr, 4 hrs, 6 hrs, 8 hrs, 12 hrs, 24 hrs, 72 hrs. PK blood samples were collected by direct vein puncture or using a venous cannula. When multiple blood sample collection were scheduled on the same day from the same participant, at the discretion of the investigator, a venous cannula was inserted and blood samples collected from the cannula to avoid multiple pricks. For the same, an intravenous (IV) cannula was placed in a forearm vein or dorsal aspect of the hand of the participants. Intravenous indwelling cannula kept in situ as long as required by injecting 0.3 mL of normal saline or heparinized saline (to prevent the cannula from clogging). Because the cannula contains stagnant blood and normal or heparinized saline, it was removed before collection of the pharmacokinetic blood sample, for the same, 0.3 mL (or as appropriate depending upon the size and length of the cannula) of normal saline/heparinized saline diluted blood was discarded prior to collection of the next PK sample. In case the indwelling cannula is blocked or does not function properly, it was removed and re-cannulation procedures performed or the blood sample was collected by direct vein puncture as per the investigator's discretion. All PK blood samples was collected or transferred (in case if collected in a syringe) in K2-EDTA blood collection tubes.

### Plasma Samples Transport

Samples were sent to laboratory for analysis of pharmacokinetic parameters. The shipment was done in dry ice by a specialized carrier. Temperatures were monitored using data logger during all transport.

### Bioanalytical Methods

After collection, the PK blood samples were centrifuged at 4000 RPM at 4° C.±2° C. for 10 minutes. The plasma was separated and transferred to labeled polypropylene tubes and stored in a freezer at −80° C.±10° C. Pharmacokinetics parameters of Urolithin A were calculated from the concentration Vs. time data using Phoenix@WinNonlin® professional software (Version 8.1.1 or higher) (Certara, Princeton, New Jersey, USA).

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic documents.

1. A composition, for admixture with water, comprising:
(a) a compound of formula (I), or a salt thereof; 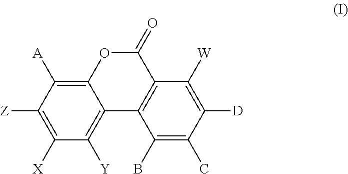
wherein:
A, B, C, D, W, X, Y and Z are each independently selected from H and OH; and
(b) maltodextrin.

2. The composition of claim 1, comprising:
(a) about 25% (w/w) to about 50% (w/w) of a compound of formula (I); and
(b) about 50% (w/w) to about 75% (w/w) maltodextrin.

3. The composition of claim 1, comprising:
(a) 1 part by weight of a compound of formula (I); and
(b) about 1 part to about 3 parts by weight maltodextrin.

4. The composition of claim 1, comprising:
(a) about 100 mg to about 2000 mg compound of formula (I), for example, urolithin A; and
(b) about 100 mg to about 6000 mg maltodextrin, for example, agglomerated maltodextrin.
wherein the weight ratio of a compound of formula (I) to maltodextrin is either:
(i) about 1 to 1 to about 1 to 3;
(ii) about 1 to 1.5 to about 1 to 2.5; or
(iii) about 1 to 2.5 to about 1 to 3.5.

5. The composition of claim 1, comprising:
(a) about 100 mg to about 2000 mg compound of formula (I), for example, urolithin A; and
(b) about 150 mg to about 5000 mg maltodextrin, for example, agglomerated maltodextrin,
wherein the weight ratio of a compound of formula (I) to maltodextrin is about 1 to 1.5 to about 1 to 2.5

6. The composition of claim 1, comprising:
(a) about 500 mg to about 1500 mg compound of formula (I), for example, urolithin A; and
(b) about 750 mg to about 3750 mg maltodextrin, for example, agglomerated maltodextrin,
wherein the weight ratio of a compound of formula (I) to maltodextrin is about 1 to 1.5 to about 1 to 2.5

7. An aqueous formulation, comprising:
(a) a compound of formula (I), or a salt thereof, as defined in claim 1; 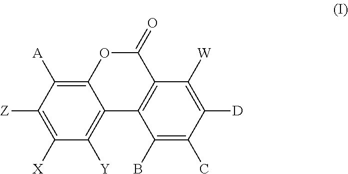
wherein:
A, B, C, D, W, X, Y and Z are each independently selected from H and OH; and
(b) maltodextrin.

8. The aqueous formulation of claim 7, comprising
(a) 1 part by weight of a compound of formula (I), or a salt thereof; and
(b) about 1 part to about 3 parts by weight maltodextrin.

9. The aqueous formulation of claim 7, comprising:
(a) about 100 mg to about 2000 mg of a compound of formula (I), or a salt thereof; and
(b) about 100 mg to about 6000 mg maltodextrin,
wherein the weight ratio of a compound of Formula (I), or a salt thereof, to maltodextrin is either:
(a) about 1 to 1 to about 1 to 3;
(b) about 1 to 1.5 to about 1 to 2.5; or
(c) about 1 to 2.5 to about 1 to 3.5.

10. The aqueous formulation of claim 7, comprising:
(a) about 100 mg/litre to about 8000 mg/litre compound of formula (I), or a salt thereof; and
(b) about 100 mg/litre to about 24 g/litre maltodextrin.

11. The aqueous formulation of claim 7, comprising either:
(i)
(a) about 0.4 millimolar to about 8.8 millimolar compound of formula (I), or a salt thereof; and
(b) about 0.06 millimolar to about 3.66 millimolar maltodextrin;
or (ii)
(a) about 0.8 millimolar to about 17.6 millimolar compound of formula (I), or a salt thereof; and
(b) about 0.12 millimolar to about 7.31 millimolar maltodextrin;
or (iii)
(a) about 0.4 millimolar to about 8.8 millimolar compound of formula (I), or a salt thereof; and
(b) about 0.24 millimolar to about 7.32 millimolar maltodextrin.

12. (canceled)

13. The aqueous formulation of claim 7, comprising either:
(i)
(a) about 0.4 millimolar to about 8.8 millimolar compound of formula (I), or a salt thereof; and
(b) about 14.7 millimolar to about 128 millimolar maltodextrin;
or (ii)
(a) about 0.8 millimolar to about 17.6 millimolar compound of formula (I), or a salt thereof; and
(b) about 29.4 millimolar to about 256 millimolar maltodextrin;
or (iii)
(a) about 1.6 millimolar to about 35.2 millimolar compound of formula (I), or a salt thereof; and
(b) about 58.8 millimolar to about 512 millimolar maltodextrin.

14. The composition of claim 1, wherein the compound of formula (I) is selected from urolithin A, urolithin B, urolithin C and/or urolithin D.

15. The composition of claim 1, wherein the maltodextrin has a DE (dextrose equivalent) of about 5 to about 15.

16. The composition of claim 1, wherein the maltodextrin is agglomerated maltodextrin.

17. A method of:
a) increasing or maintaining mitochondrial function;
b) treating, preventing, or managing a mitochondria-related condition associated with altered mitochondrial function or reduced mitochondrial density; and/or
c) treating, preventing, or managing a mitochondria-related disease,
comprising administering to a subject an effective amount of a composition of claim 1.

18. A method of enhancing muscle performance; improving, maintaining or reducing the loss of muscle function; increasing muscle strength; increasing or maintaining muscle mass; increasing or maintaining muscle recovery; inhibiting or retarding physical fatigue; enhancing working capacity and endurance; or reducing muscle fatigue, comprising administering to a subject an effective amount of a composition of claim 1.

19. (canceled)

20. (canceled)

21. (canceled)

22. (canceled)

23. A method of enhancing
a) physical performance, wherein enhancing physical performance comprises at least one effect selected from the group consisting of enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage;
b) physical recovery, wherein enhancing physical recovery comprises at least one effect selected from the group consisting of enhancing muscle recovery, enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage; and/or
c) physical endurance, wherein enhancing physical endurance comprises at least one effect selected from the group consisting of enhancing muscle recovery, enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage,
comprising administering to a subject an effective amount of a composition of claim 1.

24. (canceled)

25. (canceled)

26. (canceled)

27. A method of maintaining or enhancing skin health; reducing the impact of chronological aging on biological aging; maintaining or enhancing hair health; maintaining or enhancing immune health; maintaining or enhancing brain health; maintaining or enhancing recovery after treatment; increasing healthspan; slowing aging; or increasing longevity; comprising administering to a subject an effective amount of a composition of claim 1.

28. (canceled)

29. (canceled)

30. (canceled)

31. (canceled)

32. (canceled)

33. (canceled)

34. The composition of claim 1, wherein the compound of formula (I) is urolithin A.
